(19) United States
(12) Patent Application Publication
(10) Pub. No.: US 2016/0262524 A1
(43) Pub. Date: Sep. 15, 2016

(54) CARTRIDGE DISPENSER

(71) Applicant: Harry San Miguel, San Jose, CA (US)

(72) Inventor: Harry San Miguel, San Jose, CA (US)

(21) Appl. No.: 14/644,927

(22) Filed: Mar. 11, 2015

(51) Int. Cl. A45D 40/00; A61M 35/00; A45D 40/24

(52) U.S. Cl. CPC A45D 40/0087; A45D 40/24; A61M 35/003

(57) ABSTRACT

A cartridge dispenser filled with a plurality of cartridges having enclosed contents is disclosed. Cartridges are inserted into and retained by a cartridge interface on one end of the cartridge dispenser. The cartridge interface includes a faceted surface that matches a faceted surface on the cartridge.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates broadly to the field of dispensing cartridges from a hollow body. Specifically, the present invention relates to dispensing cartridges from a hollow body, wherein the cartridges contain cosmetic material and are capable of retaining an applicator delivering cosmetic material to a human user. More specifically still, the present invention attaches cartridges to one end of the hollow body.

2. Description of Related Art

Typically, cosmetic products are grouped together in a purse or bag, only to be retrieved individually as they are needed, then set on a surface while attention is on the application of other cosmetic products, which are also retrieved, applied, and set down on the surface. Hopefully, the surface doesn't become jammed with cosmetics, or simply is a large surface where lots of cosmetics can be spread out and easily used as needed. An attempt of organizing this chaos is a chest that contains and displays a number of cosmetic products. Cosmetic chests come in a wide variety of configurations, some being portable, and some being too large or bulky to carry easily.

Portability and organization are two problems presented by a user of multiple cosmetic products. But there is also significant issues of cross-contamination and expiration of cosmetics. Cosmetics are applied to parts of the human body, and often, in the case of eye makeup and lipstick or lip gloss, cross-contamination can occur when one person uses another person's cosmetic applicator, and germs are passed from one person to another by way of the applicator. Eye infections from applying eyeliner are a most common form of cross-contamination, but other diseases can be spread this way. Also, while cross-contamination is a threat between two users, a single user can continue to be infected by their own applicator if it is not kept free of contaminants.

Likewise, the issue of expired contents is a problem with cosmetics. Considering the cost of cosmetic products, waste is a problem of the conventional deployment of cosmetic products. In many instances, only small amounts of a particular cosmetic product are applied, but the product itself may have a shelf life that expires before the product is fully consumed. This may occur because of contamination from the user, or simply limited by the product's individual ingredient's shelf life, or their combination.

Cosmetic products are often expensive, and losing just one product out of a group of cosmetics can not only be costly, but the product can also be sorely missed by the individual who uses them. There has been no easy way to carry and apply multiple cosmetic products beyond the traditional solutions, and people who use them are most likely going to carry them around in a bag, hope they brought everything that they need, and hope that they pack it all up when they are done. The problem is compounded by the use of applicators, which often are brushes, rollers, and applicator tips of various configurations, usually dictated by the material dispensed and how it must be applied to a user. But also the problem is exacerbated by the required segregation of the individual materials, meaning that each product to be dispensed needs its own applicator.

While the problems above have been described so far in terms of cosmetic products, the user of other products, such as materials used in the medical field, faces the same problems as described above. Such medical materials must usually be maintained in a sterile environment, and kept separate before being applied to a patient or equipment used in hospital or clinical environments. These materials can include medicine, marking materials, and antiseptic.

What is needed in both applications described above is a product dispenser with multiple containers of the products that are to be applied. This dispenser is small, portable, and dispenses cartridges that can be used with a variety of different applicators, and dispenses cartridges having different contents from the other cartridges.

### BRIEF SUMMARY OF THE INVENTION

The present invention solves the problems described above by providing a cartridge dispenser body having a storage area for at least one cartridge containing cosmetic or medical material; a cartridge interface for receiving and securing a cartridge to the cartridge dispenser body; and a cartridge, the cartridge having a cover that encloses within the cartridge cosmetic or medical material and an end formed to insert into the cartridge interface to secure the cartridge to the cartridge dispenser body.

In one embodiment, the cartridge has a first faceted surface and the cartridge interface has a second faceted surface. The first faceted surface is brought into contact with the second faceted surface to retain the cartridge within the cartridge interface of the cartridge dispenser. In one embodiment, the cartridge dispenser has a first channel and a first depression formed at the end of the first channel and a second channel and a second depression formed at the end of the second channel. In one embodiment, the cartridge interface has a first protrusion formed on its interior wall at a first location and a second protrusion formed on its interior wall at a second location. In one embodiment, the cartridge is retained by the cartridge interface by matching the first and second channels of the cartridge with the first and second protrusions of the cartridge interface, and sliding the cartridge into the cartridge interface until the first and second protrusions are brought in contact with the first and second depressions of the cartridge. In one embodiment, the cartridge includes an applicator interface for receiving and retaining an applicator.

The present invention solves the problem of cross-contamination by providing a plurality of sealed cartridges inside the dispenser. Cartridges are only opened when needed, and, because only a limited amount of material is contained within a single cartridge, the present invention reduces the amount of parabens and wasted materials used in cosmetics. This also encourages the use of a fresh cartridge, thus eliminating or reducing the chance of cross-contamination between multiple users, or even a single user.

The present invention is also perfect for cosmetic kit applications, where multiple types of cosmetics are needed. Combining cosmetic materials in a single dispenser is also an excellent product for drugstores, convenience stores and the like to carry. It's an excellent product for hotels to provide guests, and can be easily distributed by vending machines in airports, train stations, malls, and the like.

The present invention is also perfect for the medical industry, art applications, industrial applications, and other uses, where the contents of the cartridges of the present invention may include marking material, medicine, coloring agents, ink, paint, adhesive, coating agents such as polyurethane, drying agents, lubricants, and the like.

Finally, in one embodiment, of this invention is to provide women a way to apply cosmetics that is cleaner than the current conventional manner. By dividing the contents of a cosmetic wand into cartridges, we delay the effects of air and other contaminates on the entire contents. We may even be able to lessen the need for parabens (the current preservatives used in cosmetics). Less contact with the air lessens the chances of clumping and contamination for cosmetics. This will results in a more effective, cleaner application of cosmetics. Some women use a safety pin to separate eye lashes when applying lumpy mascara. This invention will address issues likes this and similar issues where harmful bacteria may come in contact with the cosmetic user. In medicine the invention addresses the sterile single application of medications and ointments to the user.

Accordingly, in one embodiment, the invention also contains a cartridge dispenser system, comprising:
a) a hollow dispenser body comprising:
   i. a storage area for at least one cartridge; and
   ii. a cartridge interface for receiving and removably securing a cartridge dispenser body; and
b) a cartridge comprising:
   i. a cover;
   ii. a cosmetic or medical material positioned inside the cartridges; and
   iii. an end formed to removably attach to the cartridge interface.

### BRIEF DESCRIPTION OF THE DRAWINGS

FIG. 1 is a perspective view of the cartridge dispenser of the present invention showing a capped cartridge attached on an end of the cartridge dispenser.

FIG. 2 is a partially disassembled view of the cartridge dispenser of the present invention showing the cartridge cover removed from the cartridge attached to the cartridge dispenser as in FIG. 1.

FIG. 3 is an alternative partially disassembled view of the cartridge dispenser of the present invention showing the cartridge of FIG. 1 completely detached from the cartridge dispenser.

FIG. 4 is an alternative partially disassembled view of the cartridge dispenser of the present invention showing a cap removed from the cartridge dispenser to reveal a cartridge stored in the dispenser.

FIGS. 5-6 show various perspective views of the cartridges arranged as they would be inside the cartridge dispenser.

### DETAILED DESCRIPTION OF THE INVENTION

While this invention is susceptible to embodiment in many different forms there are shown in the drawings, and herein described in detail, specific embodiments, with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### DEFINITIONS

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

References throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures, if any, are for the purpose of illustrating certain convenient embodiments of the present invention, and are not to be considered as limitations thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “hollow cartridge dispenser body” refers to a columnar, cylinder or tube-like device that is hollow. It would be of a size sufficient to accept a cartridge in the hollow portion. That is, it has an inner diameter longer than the outer diameter of a cylindrical cartridge. The body can have a lid on the top and bottom as desired. The tube can be a system for personal use for cosmetics or medicine. In one embodiment, it is about 4″ to about 12″ long, and in another embodiment, it is about 6″ to 8″ long. In another embodiment it has a diameter of about ⅛″ to about 1″ or less. In another embodiment it has a ¼″ to ½″ diameter. The terms “storage area” refers to any part of the hollow portion of the body which can have a cartridge positioned therein.

As used herein, the terms “cartridge interface” refer to a means in the hollow of the body for attaching a cartridge. In one embodiment, it is a removably fixed attachment such that the cartridge remains secure within the body.

As used herein, a “cartridge” refers to a cylindrical-shaped container for containing a cosmetic, medicament or other liquid or solid for use by placing inside the hollow of a hollow cartridge dispenser body. The contents can be accessed through opening a lid or just be positioned inside the body. The cartridge can be removably fixed by means described elsewhere and can be removed by gravity or a device for pushing the cartridge out of the tube. One skilled in the art after review of the application could design a device for cartridge removal, the cartridge has an interface for interfacing and being removably attached in the hollow of the dispenser body. See the Drawings for an example, though one skilled in the art could design others or variations after viewing the present disclosure. In one embodiment, a cartridge is sealed and the contents are in a sterile environment.

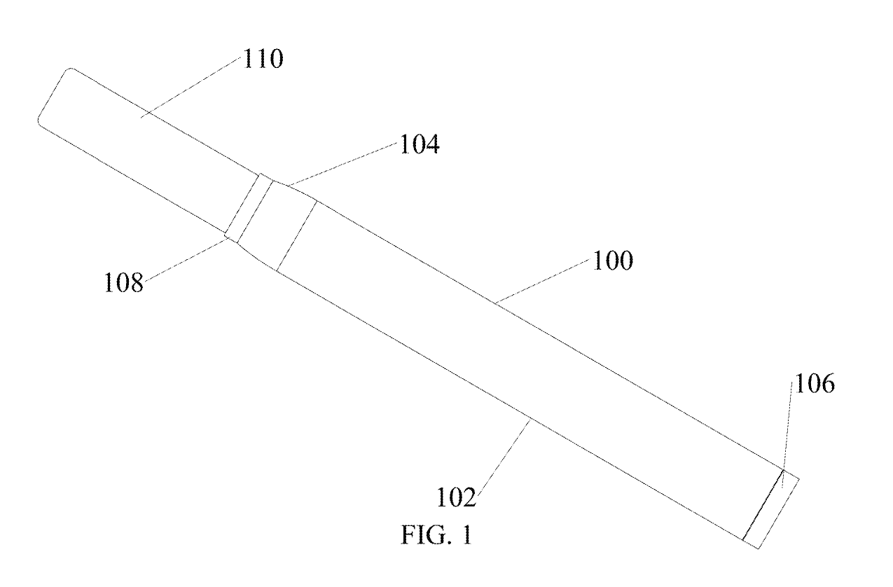

Directing attention to FIG. 1, there is shown a perspective view of the hollow cartridge dispenser body 100 having an elongated, hollow cartridge body 102 having a cartridge interface 104 at one end of body 102 and a cap 106 enclosing the opposite end of body 102. In one embodiment, body 102 serves as a storage space for multiple cartridges to be used with dispenser 100. Attached to cartridge interface 104 is cartridge base 108. Cartridge cover 110 can hold cosmetic materials in liquid, solid, or semisolid forms, as well as applicators to apply the cosmetic materials stored therein. Similarly, medical applications of the present invention can use marking material, sterilization material, or other suitable contents for medical applications. In both cosmetic and medical applications, cartridge base 108 may include a seal that keeps contents stored therein sterile.

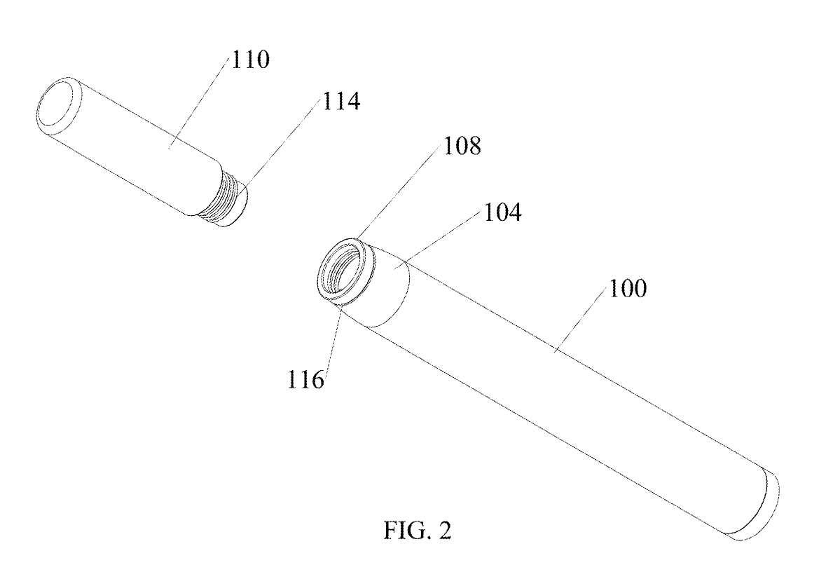

FIG. 2 shows cartridge base 108 removably retained within cartridge interface 104, but with cartridge cover 110 removed to expose cartridge circumferential edge—a threaded interface, pressure fittings of various types, or simply integral manufacture that permanently bonds or otherwise presents an applicator to cartridge base 108. Cartridge cover 110 has circumferential edge 114 that is brought proximate to or in contact with matching circumferential edge 116 on cartridge interface 104.

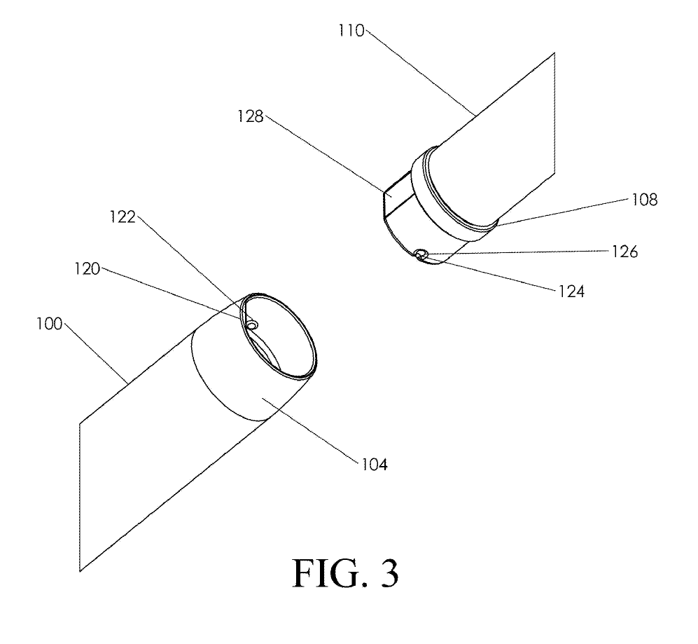

FIG. 3 shows cartridge base 108 and cartridge cover 110 retained together and removed from dispenser 100. Cartridge interface 104 includes a faceted surface 120 incorporated into a portion of an inner surface of cartridge interface 104. Located on both sides of faceted surface 120 are protrusions 122 formed integrally within the inner surface of cartridge interface 104. When cartridge base 108 is inserted into cartridge interface 104, cartridge base 108 has locating channels 124 formed in its surface that match protrusions 122 and orient cartridge base 108 in a desired configuration. Cartridge base 108 is retained in a press fit once protrusions 122 are brought in contact with depressions 126 located on opposite sides of cartridge base 108 at the end of channels 124.

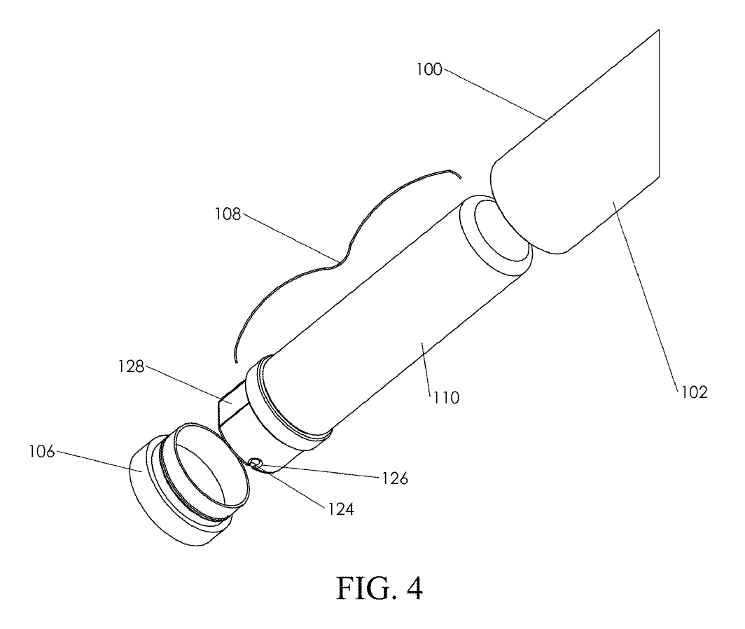

FIG. 4 illustrates cartridge dispenser 100 in a partially disassembled view with cap 106 removed to expose cartridge base 108 stored with its cartridge cover 110 inside body 102. Channel 124 and depression 126 are again both visible on one side of cartridge base 108. Also shown is facet 128, located on one portion of the circumference of cartridge base 108. Facet 128 on cartridge base 108 aligns with facet 120 in cartridge interface 104 to keep cartridge base 108 from rotating during use when retained by cartridge interface 104.

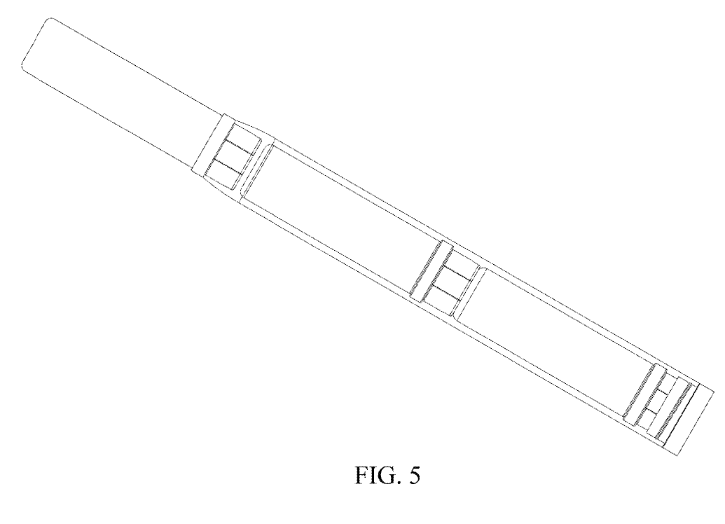

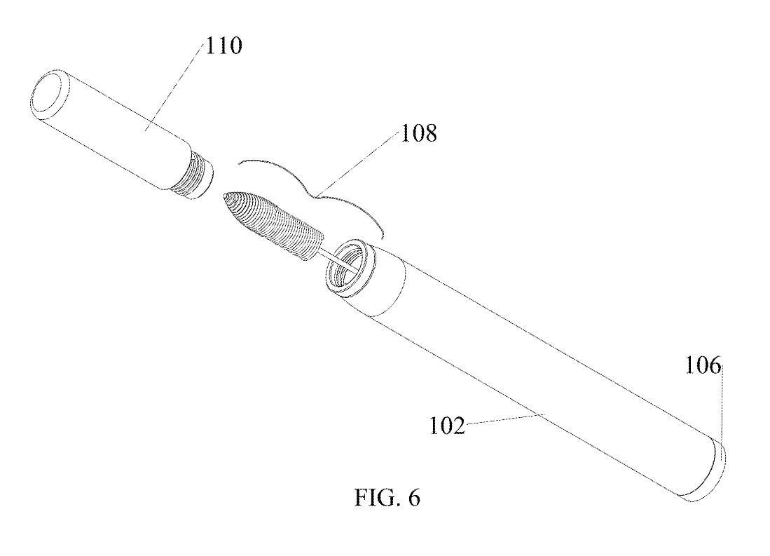

FIGS. 5 and 6 illustrate various embodiments of cartridge bases 108 and dispenser 100. As shown in FIG. 5, cartridge dispenser 100 in an embodiment contains two cartridges, but other numbers of cartridges may be utilized, as well as cartridges of varying lengths, as is desirable for maximum flexibility in choosing applicators and cosmetic materials. FIG. 6 shows cap 106 as is dimensioned to fit over the end of body 102 and also a portion of cartridge base 108.

In other embodiments of the present invention, numerous applicators are utilized in connection with cartridge base 108. For example, cartridge base 108 may include a mascara brush, eyebrow pencil, lipstick holder, brush for applying powder, and the like.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A cartridge dispenser system, comprising:
a. a cartridge dispenser body comprising:
i. a storage area for at least one cartridge; and
ii. a cartridge interface for receiving and removably securing a cartridge to the cartridge dispenser body; and
b. a cartridge, comprising:
i. a cover;
ii. a cosmetic or medical material positioned inside the cartridge; and
iii. an end formed to removably attach to the cartridge interface.

2. The cartridge dispenser of claim 1, wherein the cartridge has a first faceted surface and the cartridge interface has a second faceted surface, wherein the first faceted surface is brought into contact with the second faceted surface to retain the cartridge with the cartridge interface of the cartridge dispenser.

3. The cartridge dispenser of claim 1, wherein the cartridge dispenser has a first channel and a first depression formed at the end of the first channel and wherein the cartridge dispenser has a second channel and a second depression formed at the end of the second channel for retaining the cartridge with the cartridge interface.

4. The cartridge dispenser of claim 3, wherein the cartridge interface has a first protrusion formed on its interior wall at a first location and a second protrusion formed on its interior wall at a second location for retaining the cartridge with the cartridge interface.

5. The cartridge dispenser of claim 3, wherein the cartridge is retained by the cartridge interface by matching the first and second channels of the cartridge with the first and second protrusions of the cartridge interface, and sliding the cartridge into the cartridge interface until the first and second protrusions are brought into contact with the first and second depressions of the cartridge for retaining the cartridge with the cartridge interface.

6. The cartridge dispenser of claim 1, wherein the cartridge includes an applicator interface for receiving and retaining an applicator.

7. The cartridge dispenser system of claim 1 wherein there are a plurality of cartridges within the hollow cartridge body dispenser.

8. The cartridge dispenser system of claim 1 wherein the cartridge contains a cosmetic or medicament.

9. The cartridge dispenser system of claim 1 wherein the cartridge is sealed and the contents are sterile.
